(19) United States
(12) Patent Application Publication
(10) Pub. No.: US 2003/0156996 A1
(43) Pub. Date: Aug. 21, 2003

(54) Collection system for storage and manipulation of a sample

(76) Inventor: Jean-Francois Delorme, Montreal (CA)

Correspondence Address: OGILVY RENAULT, 1981 MCGILL COLLEGE AVENUE, SUITE 1600, MONTREAL, QC, H3A2Y3, CA

(21) Appl. No.: 10/204,451

(22) PCT Filed: Mar. 5, 2001
(86) PCT No.: PCT/CA01/00277

(51) Int. Cl. C12M 1/34; B01L 3/14

(52) U.S. Cl. 422/102; 422/99; 422/101

(57) ABSTRACT

The present invention relates to a cartridge for receiving a sample, comprising a housing, a lid capable of hermetically closing the housing and a tampered-proof system, coupled to the cartridge, to indicate to a user whether the lid has been removed from the housing, thereby allowing the user to determine if the sample has been potentially contaminated. The present invention also relates to an apparatus for storing samples, the apparatus comprising a housing, a sample receiving means provided inside the housing a sample handling device for automatically inserting and removing the samples from the sample receiving means and a user interface coupled to a control unit for controlling the sample handling device. The present invention also relates to an apparatus for automatically sampling a sample.

### BACKGROUND OF THE INVENTION

(a) Field of the Invention

The invention relates to a tamper proof sample collection system for potential automated manipulation with reduced risk of contamination, degradation or biological hazards.

(b) Description of Prior Art

It is known in the art to obtain a biological sample from a person for further analysis, such as for DNA testing, etc. These samples are typically placed on a substrate for ease of transportation and manipulation.

One known substrate is nitrocellulose paper, which has to be dried in open air when a biological substance (like blood, saliva, etc.) is applied on it. This substrate is placed in or on a container forming a device, which is typically not hermetic and does not offer a full protection against humidity, contamination, UV, degradation, etc. These devices also cause problems because it is difficult to automate the processes that use those kinds of devices. More specifically, these devices are labour-intensive, and require delicate manipulations prior to performing the analysis on the sample contained therein.

Also, after applying a biological sample on the paper, it is usually dried in open air for about 30 minutes before storing it. Such a sample might gets contaminated while drying. They could be stored immediately in a plastic bag containing a desiccant, but this is not a convenient way to handle biological sample and is not suitable for process automation.

Others substrates are used in petri dishes for different biological processes. These substrates are also sensitive to contamination. Petri dishes are still difficult to automate their manipulation.

An other issue with genetic material is the security of genetic material samples in depository, which are used in cases of forensic DNA analysis and biopharmaceutical research for example. One major concern is to prevent tampering with and the misuse of this material.

Because genetic material contains personal information about his owner and because the issue of the genetic tests can be critical (DNA test in criminal trials for example or medical diagnosis), the sample storage must be extremely secure. Actually, no real secure system protects the access to sample and no records are kept on sample access. There is no protection also on test results.

It is also known in the art to sample a part of a biological sample manually to make a test. This approach doesn't allow optimum usage of the biological sample. Also, it is impossible for a technician to recognize a sample based on the shape and the empty spots left from previous sampling. Being able to recognize a sample in that way can be a supplemental security level in the sample manipulation. Using an automated manipulation system can also reduce human intervention and reduce the risks of human errors, contamination and biological hazards. Some automated sampling systems are actually existing, but they take a sample always at the same position, which doesn't allow multiple usage of the sample.

Accordingly, it is necessary to design an automated biological sample collection system, which allows automated manipulation of biological samples, secure storage and automated sampling.

It would be highly desirable to be provided with a tamper proof biological sample collection system for potential automated manipulation with reduced risk of contamination, degradation or biological hazards.

### SUMMARY OF THE INVENTION

One aim of the present invention is to provide a tamper proof biological sample collection system for potential automated manipulation with reduced risk of contamination, degradation or biological hazards.

In accordance with the present invention there is provided a cartridge for receiving a sample, comprising:

a housing;

a lid capable of hermetically closing the housing;

a tampered-proof system, coupled to the cartridge, to indicate to a user whether the lid has been removed from the housing, thereby allowing the user to determine if the sample has been potentially contaminated, or substituted or tampered with.

The cartridge in accordance with a preferred embodiment of the present invention, wherein the tampered-proof system comprises an indicator movable into a first position indicating to a user that the cartridge has not been used, into a second position indicating to the user that the cartridge has been hermetically closed, and into a third position indicating to the user that the cartridge has been opened.

The cartridge in accordance with a preferred embodiment of the present invention, wherein the tampered-proof system is coupled to the lid.

The cartridge in accordance with a preferred embodiment of the present invention, wherein the lid comprises a sample substrate coupled thereto.

The cartridge in accordance with a preferred embodiment of the present invention, wherein the sample is a biological sample.

The cartridge in accordance with a preferred embodiment of the present invention, wherein the tampered-proof system comprises:

an indicator comprising first and second receiving positions for receiving a stopper to maintain the indicator in a first open position or a second hermetically closed position;

an element comprising first engaging means for engaging with the first and second receiving portions for moving the indicator from a first open position to the second hermetically closed position to a third open position.

The cartridge in accordance with a preferred embodiment of the present invention, wherein the tampered-proof system further comprising a means for locking said cartridge.

The cartridge in accordance with a preferred embodiment of the present invention, wherein the stopper is coupled to the element; and wherein the element further comprises second engaging means for engaging with corresponding receiving means provided on the housing, thereby hermetically closing the housing.

The cartridge in accordance with a preferred embodiment of the present invention, further comprising a desiccant.

In accordance with the present invention there is provided an apparatus for storing samples, the apparatus comprising:

a housing;

sample receiving means provided inside the housing;

a sample handling device for automatically inserting and removing the samples from the sample receiving means; and

a user interface coupled to a control unit for controlling the sample handling device.

The apparatus in accordance with a preferred embodiment of the present invention, wherein the sample is contained in a cartridge comprising:

a housing;

a lid capable of hermetically closing the housing;

a tampered-proof system, coupled to the cartridge, to indicate to a user whether the lid has been removed from the housing, thereby allowing the user to determine if the sample has been potentially contaminated.

The apparatus in accordance with a preferred embodiment of the present invention, wherein the control unit comprises a database for recording each insertion and/or removal of a sample.

The apparatus in accordance with a preferred embodiment of the present invention, wherein the sample handling device comprises:

a carrier movably mounted on the housing;

an arm coupled to the carrier; and

a grip coupled to the arm for gripping the sample.

The apparatus in accordance with a preferred embodiment of the present invention, wherein sample is contained in a cartridge comprising a bar code and/or a RF tag, and wherein the grip comprises a bar code and/or a RF scanner.

The apparatus in accordance with a preferred embodiment of the present invention, wherein the sample is a biological sample.

In accordance with the present invention there is provided an apparatus for automatically sampling a sample, the apparatus comprising:

a CPU;

a camera coupled to the CPU for scanning the sample and identifying an optimal location for sampling the sample; and

a sampler for sampling the sample at the optimal location.

The apparatus in accordance with a preferred embodiment of the present invention, wherein the sample is contained in a cartridge comprising a bar code and/or a RF tag, and wherein the grip comprises a bar code and/or a RF scanner.

The apparatus in accordance with a preferred embodiment of the present invention, wherein the sample is a biological sample.

The apparatus in accordance with a preferred embodiment of the present invention, comprising means to compare a visual signature of the sample with a visual signature previously recorded in a database, the database being located in a storage unit comprising:

a housing;

sample means provided inside the housing;

a sample handling device for automatically inserting and removing the samples from the sample receiving means;

a user interface coupled to a control unit for controlling the sample handling device wherein the control unit comprises the database.

For the purpose of the present invention the following terms are defined below.

The term “biological sample” is intended to mean a fluid sample such as blood, saliva, urine, and sperm, a non-fluid sample such as hair, nail, tissue and skin.

The term “sample” is intended to mean all biological samples as defined above and air sensitive material, light sensitive material, vaccines, pharmaceutical products, microbiological products and blood or cellular products.

The term “sample substrate” is intended to mean nitrocellulose absorbing membrane, agarose gel used in petri dishes, glass lamellas used in microscope.

The term “transaction” is intended to mean the entry, the removal or the relocalization of a sample in the storage unit, the sampling of a sample or the modification of the visual signature.

The term “visual signature” is intended to mean the numerization of the form of the sample comprising the locations of previous samples taken. That visual signature is unique for a sample.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates a front elevation view of one possible composition of a cartridge embodying the present invention.

FIG. 2 illustrates a cross-sectional view of one possible composition of the cartridge.

FIGS. 3A-C illustrate a front elevation view of the tamper proof locking and indicator mechanism. FIG. 3A illustrates the tamper proof locking and indicator mechanism in their initial position before the closing of the cartridge. FIG. 3B illustrates the tamper proof locking and indicator mechanism when the cartridge is close. FIG. 3C illustrates the tamper proof lacking and indicator mechanism after the opening of the cartridge.

FIG. 4 illustrates the automated sample storage system.

FIGS. 5A-B illustrate the horizontal mobile pick and place device.

FIG. 6 illustrates the automatic sample preprocessing system.

### DETAILED DESCRIPTION OF THE INVENTION

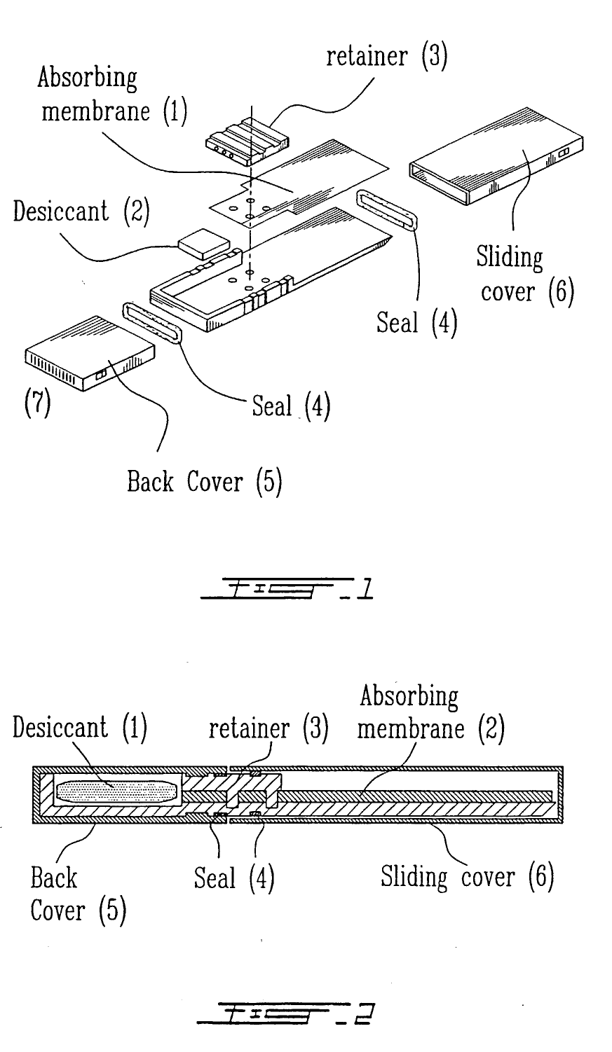

In accordance with the present invention, there is provided a tampered-proof biological sample collection cartridge. FIG. 1 shows a preferred configuration of the cartridge device according to the present invention, which contains an absorbing membrane (1) where a biological sample (like blood but not limited to) is to be applied, a desiccant (2), a desiccant retainer (3), two seals (4), a fixed back cover (5) and a sliding front cover (6). The notch on the side of the cartridge allows easy handling of the cartridge in an automated manipulation process. These notches enable gripping of the device by an automated mechanism such as robotic arm. A flat surface enables a bar code (7) to be applied to it for indexing a further storage.

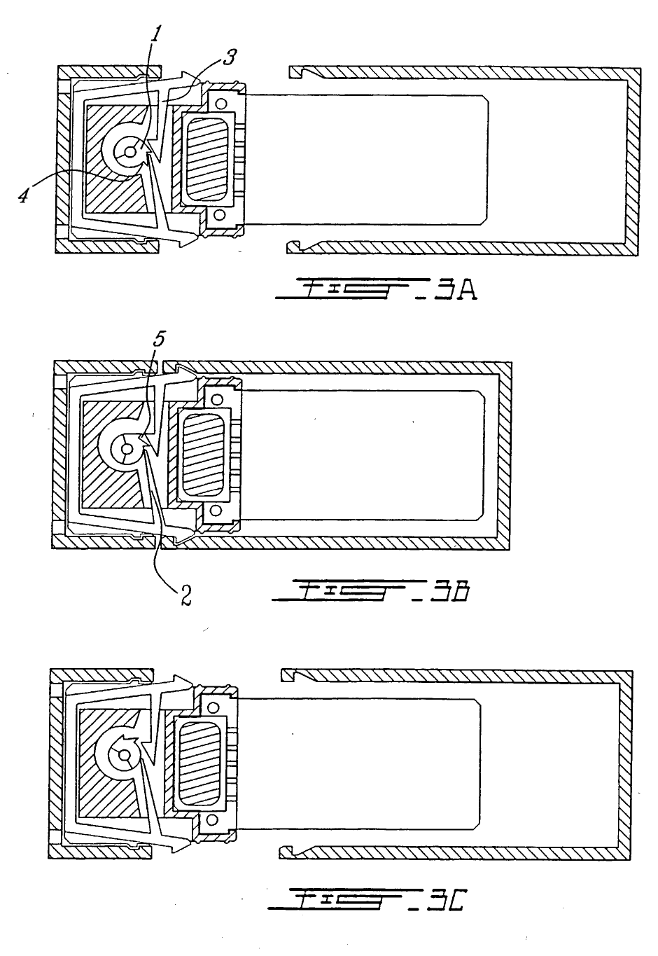

FIG. 2 illustrates a cross-sectional view of one preferred configuration of the cartridge device It incorporates a desiccant (1) to absorb humidity of the sample, an absorbing membrane (2) where the biological sample is applied, a puncture resistant, non-absorbing support that will allow easy puncturing and retrieval of a part of the absorbing membrane. The diagram shows also the desiccant retainer (3), two seals (4), a back cover (5) and a sliding front cover (6).

Depending on the use of the cartridge device, the absorbing membrane can be replaced by another sample substrate such as agarose gels and glass lamellas. If the cartridge is used for non-fluid biological samples, such hairs, the cartridge device, can has no sample substrate. If sample substrate is nitrocellulose paper, or another type of sample substrate needing drying, the device includes the desiccant retainer and the silica gel desiccant.

The cartridge device preferably includes means for closing it hermetically. Such hermetic closing protect the sample from contamination with other biological substances that can false tests results. The device also has preferably a locking device, which can be unlock only by a laboratory owning the unlocking device.

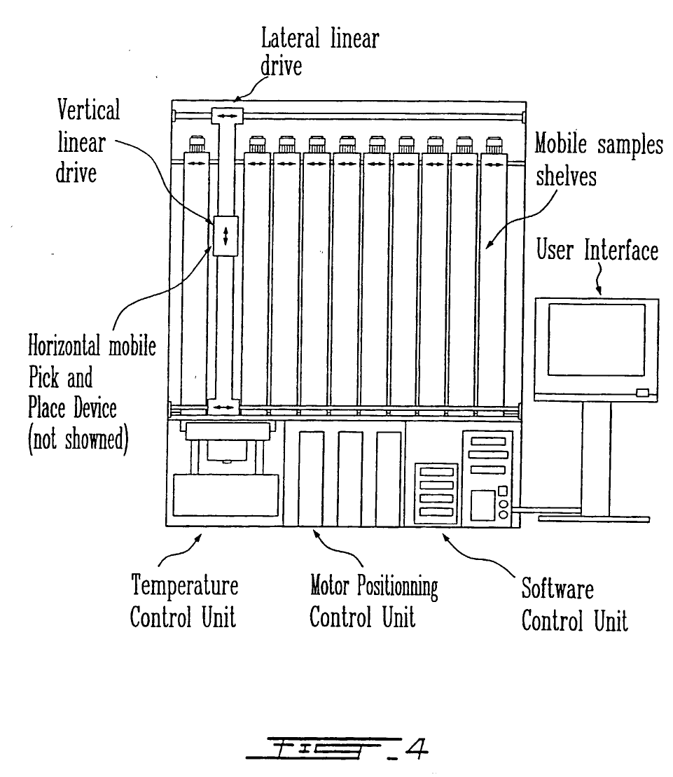

The cartridge device preferably includes a tampered-proof indicator. FIGS. 3A-C are front elevation views of the tampered-proof locking and indicator mechanism. The first diagram shows the tampered-proof mechanism when the cartridge is opened. At that time, the wheel (1) is maintained in a first opened position, such position indicating a blue color in the indicator window contained in the back cover. The blue color means that the cartridge was not used yet. The tampered-proof indicator mechanism comprises an engaging means (2) and a stopper (3) for engaging and maintaining the wheel in three distinct positions: a first open position, a second closed position and a third position indicating that the device has been opened at least once, where that third position can not be changed when it is reached.

The second diagram shows the tampered-proof mechanism when the cartridge is closed for the first time. At that time, the engaging means is engaging with the first receiving portion (4) of the wheel for moving the wheel from the first open position to a second hermetically closed position and the stopper maintain the wheel in that position. The indicator shows a green color in the indicator window contained in the back cover. The green color indicates that the cartridge was never opened.

The third diagram shows the tampered-proof mechanism when the cartridge is opened. At that time, the engaging means is engaging with the second receiving portion (5) of the wheel for moving the wheel from the second hermetically closed position to a third open position. The indicator shows a red color in the indicator window contained in the back cover. The red color indicates that the cartridge has been opened and the sample may has been tampered with that opening.

The cartridge device has a flat surface at extremity for bar code labeling. That bar code is unique for each device and it is the item that allows the sample identification and the information to be tracked in the database of the automated sample storage unit. The bar code label could be also coupled to a RF tag embedded in the plastic body of the cartridge.

Typically, this cartridge device can be used for sample analysis using PCR technique. One of these analyses is DNA analysis in forensic investigations. A blood sample from a suspect can be kept in the cartridge device for further DNA analysis, the cartridge device protecting it from possible contamination, UV degradation or sample alteration. Furthermore, the tampered-proof mechanism proves that no sample alteration or substitution had happened and guarantees that the DNA analysis is executed on the suspect's blood sample.

Another possible use of the cartridge can be for paternity testing. The user has to put a drop of blood on an absorbing paper (substrate) in the cartridge, close the cartridge and send it by messenger to the laboratory for analysis. The tampered-proof mechanism indicates to the laboratory if the cartridge had been opened during transportation, which can mean that the sample is altered. Because the samples are identified with a bar code, the confidentiality of test results is assured.

### Automated Sample Storage System

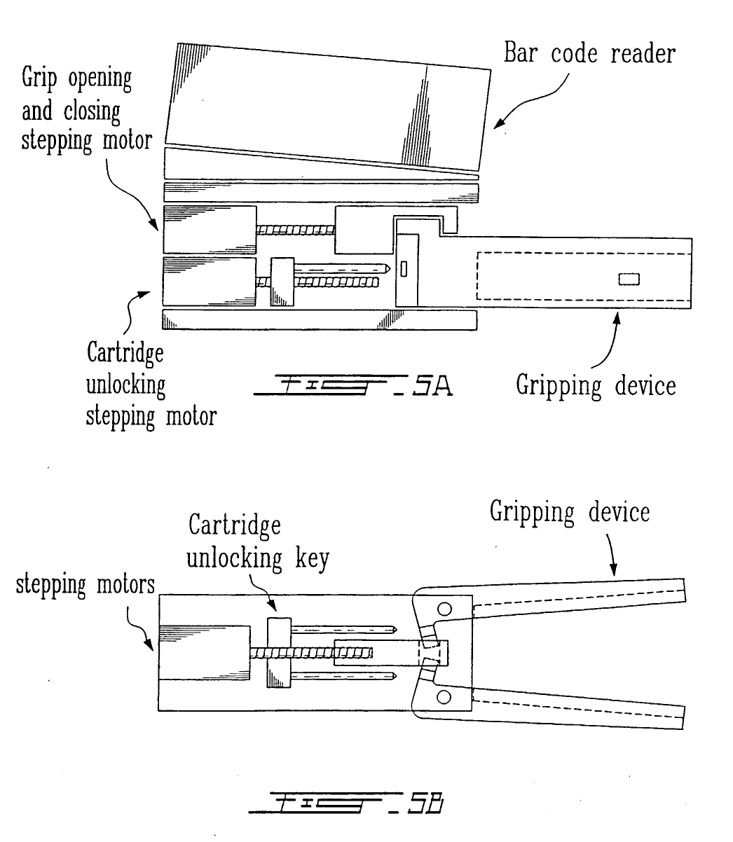

This system, as illustrated in FIG. 4 consists in a closed and temperature controlled unit comprising mobile shelves for a secure and optimal sample storage. The samples are stored in cases on the mobile shelves and the correspondence between the sample and its location in the unit is maintained in a database, using bar codes and/or RF tag to identify sample and case location.

Several levels of security exist in sample access to avoid sample misuse. The unit is closed with locked doors. To unlock these doors, the user should enter an ID and a password at the user interface to validate he is an authorized user of the system. Information about storage unit access is kept in database. There is also detectors on the door that detects an unauthorized opening of the unit. Information about unauthorized opening of the unit is kept in a separate “black box” that has is own power supply.

The sample entry in the unit is executed using an orifice on the system side. An optional support can be used to enter more than one sample at a time.

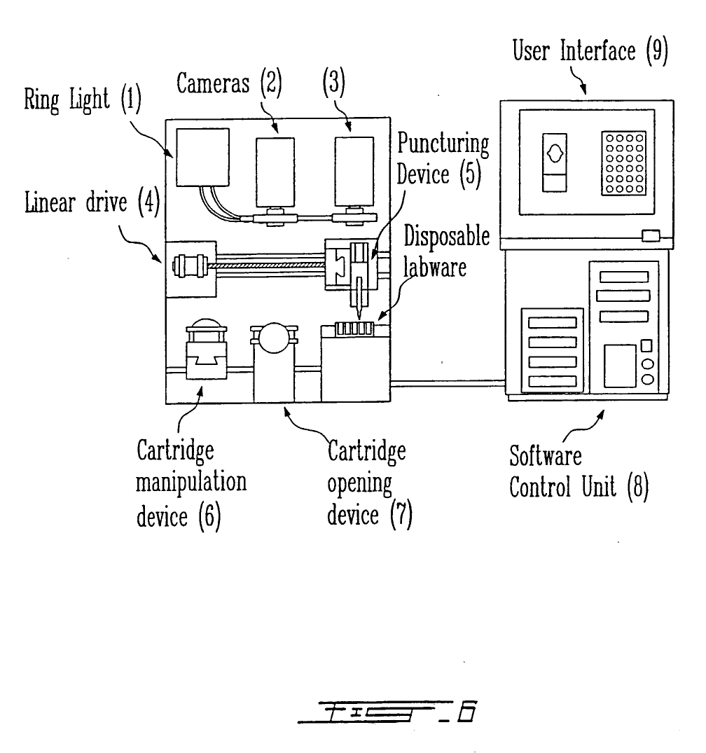

When the sample is inserted in the unit, its bar code and/or RF tag is read. If it is the first time this sample is inserted, the user can enter additional information about the sample via the user interface. A horizontal mobile pick and place device, as seen in FIGS. 5A-B, brings the sample to an empty case on a shelve of the unit. This horizontal mobile pick and place device is constituted of a robotic arm using a positioning linear two (2) axes system. The robotic arm is terminated by a grip that can handle the sample and rotate to pick or place a sample on a shelve. The grip also comprises a bar code and/or RF scanner for sample identification number before manipulation.

To remove a sample from the unit, the user shall use the user interface and request a sample in entering the sample number at the screen, or entering specific information about the sample or scanning a bar code identical to the one associated with the sample. At that time, information about the sample request is recorded in the storage unit database. The sample can also be automatically removed from the unit depending on the sample's analysis priority. Finally, after a configurable maintenance delay, the sample is placed in a disposal unit for its secure destruction. The maintenance delay can be configured through the user interface. The secure destruction prevents biological hazards and also protects biological information contained in the sample, preferably a biological sample.

All the transactions that are executed are saved on the database using the user interface software that also controls all the system's operations. This database is back up regularly on an external secure system to retrieve the information in case of system failure.

Typically, the storage system can be used for DNA-containing samples conservation. One of the possible usages is genetic repository for bioforensic. The secure storage unit database keep information about sample request and the user needs to be an authorized user to be allowed to remove a sample from the storage unit. Also, the sample is identified by a bar code, that confers confidentiality to the information related to the sample and increase the security level of the system.

### Automated Sampling System

The system can also include an automated sampling unit. This unit, as illustrated in FIG. 6 is embodied in a casing containing a ring light (1), two cameras (2-3), a linear drive (4), a puncturing device (5), a manipulation device (6), a sterilization unit, a visual inspection station, a bar code printer, an opening device (7), a software control unit (8) and a user interface (9).

The system is used to automatically take a sample from another sample that can be contained in a plurality of different substrates. The sample is preferably a DNA-containing sample.

The user can configure the system, using the user interface, in order to adjust the system for a plurality of containers. These containers can be, but not limited to, microplates, Eppendorf tubes and test tubes.

When a user want to take a sample from another sample, a container is first putted under the right camera and a picture of the container is taken. The system uses the picture to verify that the container is empty. Then the material, which is preferably the cartridge previously described, is inserted by the manipulation device and placed under the left camera. If the material is a cartridge, the opening device open it. A picture of the material to be sample is taken and analyzed.

Using the picture of the material, the artificial vision technique identify where on the material there is substance to be sample, where there is no substance and where a sample was previously taken. The artificial vision technique also optimizes the sample localization. The optimization method uses mathematical algorithms.

When the optimal sample localization is determined, the puncturing device at that position takes the sample. The sample is then putted in the container and a picture, with the right camera, is taken to validate the presence of the sample in the container. If another sample is needed, the optimization method is executed again. If not, the puncturing device is sterilized in the sterilization unit and the container with the sample is removed from the sampling unit.

The sampling system is preferably coupled with the sample storage unit previously described. When the sampling unit takes a picture of the material to be sampled, it compares the visual signature of the material with the one previously saved in the storage unit database. After the sample is taken, the new visual signature is replaced in the storage unit database. The storage unit and the sampling unit communicate together using TCP/IP protocol and SQL queries.

While the invention has been described with particular reference to the illustrated embodiment, it will be understood that numerous modifications thereto will appear to those skilled in the art. Accordingly, the above description and accompanying drawings should be taken as illustrative of the invention and not in a limiting sense.

What is claimed is:

1. A cartridge for receiving a sample, comprising:
a housing;
a lid capable of hermetically closing the housing;
a tampered-proof system, coupled to the cartridge, to indicate to a user whether the lid has been removed from the housing, thereby allowing the user to determine if the sample has been potentially contaminated or substituted or tampered with.

2. A cartridge as claimed in claim 1 wherein the tampered-proof system comprises an indicator movable into a first position indicating to a user that the cartridge has not been used, into a second position indicating to the user that the cartridge has been hermetically closed, and into a third position indicating to the user that the cartridge has been opened.

3. A cartridge as claimed in claim 1 wherein the tampered-proof system is coupled to the lid.

4. A cartridge as claimed in claim 1 wherein the lid comprises a sample substrate coupled thereto.

5. A cartridge as claimed in claim 1 wherein the sample is a biological sample.

6. A cartridge as claimed in claim 3 wherein the tampered-proof system comprises:
an indicator comprising first and second receiving positions for receiving a stopper to maintain the indicator in a first open position or a second hermetically closed position;
an element comprising first engaging means for engaging with the first and second receiving portions for moving the indicator from a first open position to the second hermetically closed position to a third open position

7. A cartridge as claimed in claim 6 wherein the stopper is coupled to the element; and wherein the element further comprises second engaging means for engaging with corresponding receiving means provided on the housing, thereby hermetically closing the housing.

8. A cartridge as claimed in claim 1, wherein the tampered-proof system further comprising a means for locking said cartridge.

9. A cartridge as claimed in claim 1 further comprising a desiccant.

10. An apparatus for storing samples, said apparatus comprising:
a housing;
sample receiving means provided inside the housing;
a sample handling device for automatically inserting and removing the samples from the sample receiving means; and
a user interface coupled to a control unit for controlling the sample handling device.

11. An apparatus as claimed in claim 10 wherein the sample is contained in a cartridge comprising:
a housing;
a lid capable of hermetically closing the housing;
a tampered-proof system, coupled to the cartridge, to indicate to a user whether the lid has been removed from the housing, thereby allowing the user to determine if the sample has been potentially contaminated or substituted or tampered with.

12. An apparatus as claimed in claim 10 wherein the control unit comprises a database for recording each insertion and/or removal of a sample.

13. An apparatus as claimed in claim 10 wherein the sample handling device comprises:
a carrier movably mounted on the housing;
an arm coupled to the carrier; and
a grip coupled to the arm for gripping the sample.

14. An apparatus as claimed in claim 13 wherein sample is contained in a cartridge comprising a bar code and/or a RF tag, and wherein the grip comprises a bar code and/or a RF scanner.

15. An apparatus as claimed in claim 14 wherein the sample is a biological sample.

16. An apparatus for automatically sampling a sample, said apparatus comprising:
a CPU;
a camera coupled to the CPU for scanning the sample and identifying an optimal location for sampling the sample; and
a sampler for sampling the sample at the optimal location.

17. An apparatus as claimed in claim 16 wherein the sample is contained in a cartridge comprising a bar code and/or a RF tag, and wherein the grip comprises a bar code and/or a RF scanner.

18. An apparatus as claimed in claim 17 wherein the sample is a biological sample.

19. An apparatus as claimed in claim 16 comprising means to compare a visual signature of the sample with a visual signature previously recorded in a database, said database being located in a storage unit comprising:
a housing;
sample means provided inside the housing;
a sample handling device for automatically inserting and removing the samples from the sample receiving means;
a user interface coupled to a control unit for controlling the sample handling device wherein the control unit comprises the database.
